SEQUENCE LISTING (16 sequences)

SEQ ID NO: 1 - protein, 616 aa, synthetic construct
  REGION 1..616: note = synthetic_LeuA AA (616 aa)
MSPNDAFISAPAKIETPVGPRNEGQPAWNKQRGSSMPVNRYMPFEVEVEDISLPDRTWPDKKITVAPQWCAVDLRDGNQALIDPMSPERKRRMFELLVQMGFKEIEVGFPSASQTDFDFVREIIEKGMIPDDVTIQVLVQAREHLIRRTFEACEGAKNVIVHFYNSTSILQRNVVFRMDKVQVKKLATDAAELIKTIAQDYPDTNWRWQYSPESFTGTEVEYAKEVVDAVVEVMDPTPENPMIINLPSTVEMITPNVYADSIEWMHRNLNRRDSIILSLHPHNDRGTGVGAAELGYMAGADRIEGCLFGNGERTGNVCLVTLALNMLTQGVDPQLDFTDIRQIRSTVEYCNQLRVPERHPYGGDLVFTAFSGSHQDAVNKGLDAMAAKVQPGASSTEVSWEQLRDTEWEVPYLPIDPKDVGRDYEAVIRVNSQSGKGGVAYIMKTDHGLQIPRSMQVEFSTVVQNVTDAEGGEVNSKAMWDIFATEYLERTAPVEQIALRVENAQTENEDASITAELIHNGKDVTVDGRGNGPLAAYANALEKLGIDVEIQEYNQHARTSGDDAEAAAYVLAEVNGRKVWGVGIAGSITYASLKAVTSAVNRALDVNHEAVLAGGV

SEQ ID NO: 2 - DNA, 1851 bp, synthetic construct
  misc_feature 1..1851: note = synthetic_leuA NT
  source 1..1851: mol_type = other DNA
atgtctcctaacgatgcattcatctccgcacctgccaagatcgaaaccccagttgggcctcgcaacgaaggccagccagcatggaataagcagcgtggctcctcaatgccagttaaccgctacatgcctttcgaggttgaggtagaagatatttctctgccggaccgcacttggccagataaaaaaatcaccgttgcacctcagtggtgtgctgttgacctgcgtgacggcaaccaggctctgattgatccgatgtctcctgagcgtaagcgccgcatgtttgagctgctggttcagatgggcttcaaagaaatcgaggtcggtttcccttcagcttcccagactgattttgatttcgttcgtgagatcatcgaaaagggcatgatccctgacgatgtcaccattcaggttctggttcaggctcgtgagcacctgattcgccgtacttttgaagcttgcgaaggcgcaaaaaacgttatcgtgcacttctacaactccacctccatcctgcagcgcaacgtggtgttccgcatggacaaggtgcaggtgaagaagctggctaccgatgccgctgaactaatcaagaccatcgctcaggattacccagacaccaactggcgctggcagtactcccctgagtccttcaccggcactgaggttgagtacgccaaggaagttgtggacgcagttgttgaggtcatggatccaactcctgagaacccaatgatcatcaacctgccttccaccgttgagatgatcacccctaacgtttacgcagactccattgaatggatgcaccgcaatctaaaccgtcgtgattccattatcctgtccctgcacccgcacaatgaccgtggcaccggcgttggcgcagctgagctgggctacatggctggcgctgaccgcatcgaaggctgcctgttcggcaacggcgagcgcaccggcaacgtctgcctggtcaccctggcactgaacatgctgacccagggcgttgaccctcagctggacttcaccgatatacgccagatccgcagcaccgttgaatactgcaaccagctgcgcgttcctgagcgccacccatacggcggtgacctggtcttcaccgctttctccggttcccaccaggacgctgtgaacaagggtctggacgccatggctgccaaggttcagccaggtgctagctccactgaagtttcttgggagcagctgcgcgacaccgaatgggaggttccttacctgcctatcgatccaaaggatgtcggtcgcgactacgaggctgttatccgcgtgaactcccagtccggcaagggcggcgttgcttacatcatgaagaccgatcacggtctgcagatccctcgctccatgcaggttgagttctccaccgttgtccagaacgtcaccgacgctgagggcggcgaggtcaactccaaggcaatgtgggatatcttcgccaccgagtacctggagcgcaccgcaccagttgagcagatcgcgctgcgcgtcgagaacgctcagaccgaaaacgaggatgcatccatcaccgccgagctcatccacaacggcaaggacgtcaccgtcgatggccgcggcaacggcccactggccgcttacgccaacgcgctggagaagctgggcatcgacgttgagatccaggaatacaaccagcacgcccgcacctcgggcgacgatgcagaagcagccgcctacgtgctggctgaggtcaacggccgcaaggtctggggcgtcggcatcgctggctccatcacctacgcttcgctgaaggcagtgacctccgccgtaaaccgcgcgctggacgtcaaccacgaggcagtcctggctggcggcgtttaa

SEQ ID NO: 3 - protein, 616 aa, synthetic construct
  REGION 1..616: note = synthetic_LeuA P247C AA (616 aa)
MSPNDAFISAPAKIETPVGPRNEGQPAWNKQRGSSMPVNRYMPFEVEVEDISLPDRTWPDKKITVAPQWCAVDLRDGNQALIDPMSPERKRRMFELLVQMGFKEIEVGFPSASQTDFDFVREIIEKGMIPDDVTIQVLVQAREHLIRRTFEACEGAKNVIVHFYNSTSILQRNVVFRMDKVQVKKLATDAAELIKTIAQDYPDTNWRWQYSPESFTGTEVEYAKEVVDAVVEVMDPTPENPMIINLCSTVEMITPNVYADSIEWMHRNLNRRDSIILSLHPHNDRGTGVGAAELGYMAGADRIEGCLFGNGERTGNVCLVTLALNMLTQGVDPQLDFTDIRQIRSTVEYCNQLRVPERHPYGGDLVFTAFSGSHQDAVNKGLDAMAAKVQPGASSTEVSWEQLRDTEWEVPYLPIDPKDVGRDYEAVIRVNSQSGKGGVAYIMKTDHGLQIPRSMQVEFSTVVQNVTDAEGGEVNSKAMWDIFATEYLERTAPVEQIALRVENAQTENEDASITAELIHNGKDVTVDGRGNGPLAAYANALEKLGIDVEIQEYNQHARTSGDDAEAAAYVLAEVNGRKVWGVGIAGSITYASLKAVTSAVNRALDVNHEAVLAGGV

SEQ ID NO: 4 - DNA, 1851 bp, synthetic construct
  misc_feature 1..1851: note = synthetic_LeuA P247C NT
  source 1..1851: mol_type = other DNA
atgtctcctaacgatgcattcatctccgcacctgccaagatcgaaaccccagttgggcctcgcaacgaaggccagccagcatggaataagcagcgtggctcctcaatgccagttaaccgctacatgcctttcgaggttgaggtagaagatatttctctgccggaccgcacttggccagataaaaaaatcaccgttgcacctcagtggtgtgctgttgacctgcgtgacggcaaccaggctctgattgatccgatgtctcctgagcgtaagcgccgcatgtttgagctgctggttcagatgggcttcaaagaaatcgaggtcggtttcccttcagcttcccagactgattttgatttcgttcgtgagatcatcgaaaagggcatgatccctgacgatgtcaccattcaggttctggttcaggctcgtgagcacctgattcgccgtacttttgaagcttgcgaaggcgcaaaaaacgttatcgtgcacttctacaactccacctccatcctgcagcgcaacgtggtgttccgcatggacaaggtgcaggtgaagaagctggctaccgatgccgctgaactaatcaagaccatcgctcaggattacccagacaccaactggcgctggcagtactcccctgagtccttcaccggcactgaggttgagtacgccaaggaagttgtggacgcagttgttgaggtcatggatccaactcctgagaacccaatgatcatcaacctgtgttccaccgttgagatgatcacccctaacgtttacgcagactccattgaatggatgcaccgcaatctaaaccgtcgtgattccattatcctgtccctgcacccgcacaatgaccgtggcaccggcgttggcgcagctgagctgggctacatggctggcgctgaccgcatcgaaggctgcctgttcggcaacggcgagcgcaccggcaacgtctgcctggtcaccctggcactgaacatgctgacccagggcgttgaccctcagctggacttcaccgatatacgccagatccgcagcaccgttgaatactgcaaccagctgcgcgttcctgagcgccacccatacggcggtgacctggtcttcaccgctttctccggttcccaccaggacgctgtgaacaagggtctggacgccatggctgccaaggttcagccaggtgctagctccactgaagtttcttgggagcagctgcgcgacaccgaatgggaggttccttacctgcctatcgatccaaaggatgtcggtcgcgactacgaggctgttatccgcgtgaactcccagtccggcaagggcggcgttgcttacatcatgaagaccgatcacggtctgcagatccctcgctccatgcaggttgagttctccaccgttgtccagaacgtcaccgacgctgagggcggcgaggtcaactccaaggcaatgtgggatatcttcgccaccgagtacctggagcgcaccgcaccagttgagcagatcgcgctgcgcgtcgagaacgctcagaccgaaaacgaggatgcatccatcaccgccgagctcatccacaacggcaaggacgtcaccgtcgatggccgcggcaacggcccactggccgcttacgccaacgcgctggagaagctgggcatcgacgttgagatccaggaatacaaccagcacgcccgcacctcgggcgacgatgcagaagcagccgcctacgtgctggctgaggtcaacggccgcaaggtctggggcgtcggcatcgctggctccatcacctacgcttcgctgaaggcagtgacctccgccgtaaaccgcgcgctggacgtcaaccacgaggcagtcctggctggcggcgtttaa

SEQ ID NO: 5 - protein, 616 aa, synthetic construct
  REGION 1..616: note = synthetic_LeuA P247C R558H G561D AA (616 aa)
MSPNDAFISAPAKIETPVGPRNEGQPAWNKQRGSSMPVNRYMPFEVEVEDISLPDRTWPDKKITVAPQWCAVDLRDGNQALIDPMSPERKRRMFELLVQMGFKEIEVGFPSASQTDFDFVREIIEKGMIPDDVTIQVLVQAREHLIRRTFEACEGAKNVIVHFYNSTSILQRNVVFRMDKVQVKKLATDAAELIKTIAQDYPDTNWRWQYSPESFTGTEVEYAKEVVDAVVEVMDPTPENPMIINLCSTVEMITPNVYADSIEWMHRNLNRRDSIILSLHPHNDRGTGVGAAELGYMAGADRIEGCLFGNGERTGNVCLVTLALNMLTQGVDPQLDFTDIRQIRSTVEYCNQLRVPERHPYGGDLVFTAFSGSHQDAVNKGLDAMAAKVQPGASSTEVSWEQLRDTEWEVPYLPIDPKDVGRDYEAVIRVNSQSGKGGVAYIMKTDHGLQIPRSMQVEFSTVVQNVTDAEGGEVNSKAMWDIFATEYLERTAPVEQIALRVENAQTENEDASITAELIHNGKDVTVDGRGNGPLAAYANALEKLGIDVEIQEYNQHAHTSDDDAEAAAYVLAEVNGRKVWGVGIAGSITYASLKAVTSAVNRALDVNHEAVLAGGV

SEQ ID NO: 6 - DNA, 1851 bp, synthetic construct
  misc_feature 1..1851: note = synthetic_LeuA P247C R558H G561D NT
  source 1..1851: mol_type = other DNA
atgtctcctaacgatgcattcatctccgcacctgccaagatcgaaaccccagttgggcctcgcaacgaaggccagccagcatggaataagcagcgtggctcctcaatgccagttaaccgctacatgcctttcgaggttgaggtagaagatatttctctgccggaccgcacttggccagataaaaaaatcaccgttgcacctcagtggtgtgctgttgacctgcgtgacggcaaccaggctctgattgatccgatgtctcctgagcgtaagcgccgcatgtttgagctgctggttcagatgggcttcaaagaaatcgaggtcggtttcccttcagcttcccagactgattttgatttcgttcgtgagatcatcgaaaagggcatgatccctgacgatgtcaccattcaggttctggttcaggctcgtgagcacctgattcgccgtacttttgaagcttgcgaaggcgcaaaaaacgttatcgtgcacttctacaactccacctccatcctgcagcgcaacgtggtgttccgcatggacaaggtgcaggtgaagaagctggctaccgatgccgctgaactaatcaagaccatcgctcaggattacccagacaccaactggcgctggcagtactcccctgagtccttcaccggcactgaggttgagtacgccaaggaagttgtggacgcagttgttgaggtcatggatccaactcctgagaacccaatgatcatcaacctgtgttccaccgttgagatgatcacccctaacgtttacgcagactccattgaatggatgcaccgcaatctaaaccgtcgtgattccattatcctgtccctgcacccgcacaatgaccgtggcaccggcgttggcgcagctgagctgggctacatggctggcgctgaccgcatcgaaggctgcctgttcggcaacggcgagcgcaccggcaacgtctgcctggtcaccctggcactgaacatgctgacccagggcgttgaccctcagctggacttcaccgatatacgccagatccgcagcaccgttgaatactgcaaccagctgcgcgttcctgagcgccacccatacggcggtgacctggtcttcaccgctttctccggttcccaccaggacgctgtgaacaagggtctggacgccatggctgccaaggttcagccaggtgctagctccactgaagtttcttgggagcagctgcgcgacaccgaatgggaggttccttacctgcctatcgatccaaaggatgtcggtcgcgactacgaggctgttatccgcgtgaactcccagtccggcaagggcggcgttgcttacatcatgaagaccgatcacggtctgcagatccctcgctccatgcaggttgagttctccaccgttgtccagaacgtcaccgacgctgagggcggcgaggtcaactccaaggcaatgtgggatatcttcgccaccgagtacctggagcgcaccgcaccagttgagcagatcgcgctgcgcgtcgagaacgctcagaccgaaaacgaggatgcatccatcaccgccgagctcatccacaacggcaaggacgtcaccgtcgatggccgcggcaacggcccactggccgcttacgccaacgcgctggagaagctgggcatcgacgttgagatccaggaatacaaccagcacgcccacacctcggatgacgatgcagaagcagccgcctacgtgctggctgaggtcaacggccgcaaggtctggggcgtcggcatcgctggctccatcacctacgcttcgctgaaggcagtgacctccgccgtaaaccgcgcgctggacgtcaaccacgaggcagtcctggctggcggcgtttaa

SEQ ID NO: 7 - DNA, 30 bp, synthetic construct
  misc_feature 1..30: note = synthetic_pCR-leuA_F
  source 1..30: mol_type = other DNA
ctaatctcgaggtcacccatgtctcctaac

SEQ ID NO: 8 - DNA, 27 bp, synthetic construct
  misc_feature 1..27: note = synthetic_pCR-leuA_R
  source 1..27: mol_type = other DNA
ggctggcggcgtttaaaaccggttgat

SEQ ID NO: 9 - DNA, 22 bp, synthetic construct
  misc_feature 1..22: note = synthetic_leuA_sequencing_F
  source 1..22: mol_type = other DNA
aacacgaccggcatcccgtcgc

SEQ ID NO: 10 - DNA, 23 bp, synthetic construct
  misc_feature 1..23: note = synthetic_leuA_sequencing_R
  source 1..23: mol_type = other DNA
aaatcatttgagaaaactcgagg

SEQ ID NO: 11 - DNA, 45 bp, synthetic construct
  misc_feature 1..45: note = synthetic_leuA_P247C_Up_F
  source 1..45: mol_type = other DNA
gtgaattcgagctcggtacccaaatcatttgagaaaactcgaggc

SEQ ID NO: 12 - DNA, 45 bp, synthetic construct
  misc_feature 1..45: note = synthetic_leuA_P247C_Up_R
  source 1..45: mol_type = other DNA
ggtgatcatctcaacggtggaacacaggttgatgatcattgggtt

SEQ ID NO: 13 - DNA, 45 bp, synthetic construct
  misc_feature 1..45: note = synthetic_leuA_P247C_Down_F
  source 1..45: mol_type = other DNA
aacccaatgatcatcaacctgtgttccaccgttgagatgatcacc

SEQ ID NO: 14 - DNA, 42 bp, synthetic construct
  misc_feature 1..42: note = synthetic_leuA_P247C_Down_R
  source 1..42: mol_type = other DNA
ggtcgactctagaggatccccaagaaggcaacatcggacagc

SEQ ID NO: 15 - DNA, 21 bp, synthetic construct
  misc_feature 1..21: note = synthetic_leuA_sequencing_R
  source 1..21: mol_type = other DNA
atccattcaatggagtctgcg

SEQ ID NO: 16 - protein, 581 aa, synthetic construct
  REGION 1..581: note = synthetic_LeuA AA (581 aa)
MPVNRYMPFEVEVEDISLPDRTWPDKKITVAPQWCAVDLRDGNQALIDPMSPERKRRMFELLVQMGFKEIEVGFPSASQTDFDFVREIIEKGMIPDDVTIQVLVQAREHLIRRTFEACEGAKNVIVHFYNSTSILQRNVVFRMDKVQVKKLATDAAELIKTIAQDYPDTNWRWQYSPESFTGTEVEYAKEVVDAVVEVMDPTPENPMIINLPSTVEMITPNVYADSIEWMHRNLNRRDSIILSLHPHNDRGTGVGAAELGYMAGADRIEGCLFGNGERTGNVCLVTLALNMLTQGVDPQLDFTDIRQIRSTVEYCNQLRVPERHPYGGDLVFTAFSGSHQDAVNKGLDAMAAKVQPGASSTEVSWEQLRDTEWEVPYLPIDPKDVGRDYEAVIRVNSQSGKGGVAYIMKTDHGLQIPRSMQVEFSTVVQNVTDAEGGEVNSKAMWDIFATEYLERTAPVEQIALRVENAQTENEDASITAELIHNGKDVTVDGRGNGPLAAYANALEKLGIDVEIQEYNQHARTSGDDAEAAAYVLAEVNGRKVWGVGIAGSITYASLKAVTSAVNRALDVNHEAVLAGGV

(19) United States
(12) Patent Application Publication
(10) Pub. No.: US 2026/0159862 A1
(43) Pub. Date: Jun. 11, 2026

(54) Isopropylmalate synthase polypeptide variant and a method for producing L-leucine using the same

(71) Applicant: CJ CHEILJEDANG CORPORATION, Seoul (KR)

(72) Inventors: Hayun LEE, Seoul (KR); Ju Eun KIM, Seoul (KR); Jihyun SHIM, Seoul (KR); Ji Hye LEE, Seoul (KR); Sung Gun LEE, Seoul (KR)

(73) Assignee: CJ CHEILJEDANG CORPORATION, Seoul (KR)

(21) Appl. No.: 19/451,334

(22) Filed: Jan. 16, 2026

Related U.S. Application Data
(62) Division of application No. 17/760,462, now Pat. No. 12,553,069.

(30) Foreign Application Priority Data
May 20, 2020 (KR) 10-2020-0060578

(51) Int. Cl. C12P 13/06; C12N 9/10; C12N 15/77

(52) U.S. Cl. CPC C12P 13/06; C12N 9/1025; C12N 15/77; C12Y 203/03013; C12N 2800/101

(57) ABSTRACT

The present application relates to: a novel mutant polypeptide having isopropylmalate synthase activity; and a method for producing L-leucine by using same. L-leucine can be produced at high yield by using the mutant polypeptide according to an embodiment.

### CROSS REFERENCE TO RELATED APPLICATION(S)

The present application claims the benefit of Korean patent application No. 10-2020-0060578 filed on May 20, 2020, and the entire contents disclosed in the documents of the corresponding Korean patent applications are incorporated as a part of the present specification.

### TECHNICAL FIELD

The present application relates to a novel mutated polypeptide having an isopropylmalate synthase activity; a polynucleotide encoding the same; a vector comprising the polynucleotide; a microorganism comprising the mutated polypeptide, the polynucleotide, the vector, or a combination thereof; and a method for producing L-leucine by culturing the microorganism.

### BACKGROUND ART

L-Amino acids are industrially produced by fermentation methods using amino acid-producing bacteria belonging to the coryneform bacteria or the family Enterobacteriaceae which has an ability to produce an L-amino acid. As these amino acid-producing bacteria, strains isolated from the nature as well as artificial mutants of such strains and recombinant strains in which L-amino acid biosynthesis enzyme is enhanced by genetic recombination are used in order to improve the productivity.

Among the L-amino acids, L-leucine is a type of essential amino acid, one which is expensive and widely used in medicines, foods, feed additives, industrial chemicals, and the like. Further, L-leucine is mainly produced using microorganisms. The fermentation production of L-leucine is mainly carried out through a microorganism of the genus Escherichia or a microorganism of the genus Corynebacterium, which is known to biosynthesize 2-ketoisocaproate as a precursor from pyruvic acid though several steps. However, the enzymes involved in L-leucine biosynthesis cause feedback inhibition by the final product, i.e., L-leucine or a derivative thereof, thus making it difficult to perform large scale industrial production of L-leucine.

Therefore, the present inventors have found that a mutant strain obtained by altering a specific position of isopropylmalate synthase can optimize enzyme activity and thus can be used to produce L-leucine in high yield, thereby completing the present invention.

### PRIOR ART LITERATURE

### Patent Literature

(Patent Literature 1) US 2018-0251772 A1

### DISCLOSURE

### Technical Problem

An object of the present application is to provide a mutated polypeptide having isopropylmalate synthase activity in which a proline amino acid residue at position 247 in the amino acid sequence of SEQ ID NO: 1 is substituted with an amino acid residue other than proline. Another object of the present application is to provide a polynucleotide encoding the mutated polypeptide.

Another object of the present application is to provide a vector comprising the polynucleotide.

Another object of the present application is to provide a microorganism comprising at least one selected from the group consisting of the mutated polypeptide, the polynucleotide, and the vector comprising the polynucleotide.

Another object of the present application is to provide a method for producing L-leucine comprising the step of culturing the microorganism in a medium.

Another object of the present application is to provide a composition for producing L-leucine comprising at least one selected from the group consisting of the mutated polypeptide, a polynucleotide encoding the mutated polypeptide, a vector comprising the polynucleotide, and the microorganism (for example, a mutated polypeptide of the present application, a polynucleotide of the present application, and/or a microorganism (recombinant cell) comprising a vector of the present application).

Another object of the present application is to provide a use of at least one selected from the group consisting of the mutated polypeptide, a polynucleotide encoding the mutated polypeptide, a vector comprising the polynucleotide, and the microorganism (for example, a mutated polypeptide of the present application, a polynucleotide of the present application, and/or a microorganism (recombinant cell) comprising a vector of the present application) for producing L-leucine.

### Technical Solution

In one aspect of present application, there is provided a mutated polypeptide having an isopropylmalate synthase activity in which an amino acid residue at position 247 from the N-terminal in the amino acid sequence of the isopropylmalate synthase protein of SEQ ID NO: 1 or at a position corresponding thereto is substituted with another amino acid residue.

In this specification, the term “isopropylmalate synthase (2-isopropylmalate synthase, α-isopropylmalate synthase)” refers to an enzyme that catalyzes the condensation of acetyl group of acetyl-CoA and 2-ketoisovalerate (3-methyl-2-oxobutanoate, 2-exoisovalerate) to convert it to isopropylmalate (3-carboxy-3-hydroxy-4-methylpentanoate), which is a precursor of L-leucine. The isopropylmalate synthase may comprise the enzyme having the conversion activity, regardless of an origin of a microorganism. For example, the isopropylmalate synthase may be an enzyme derived from a microorganism of the genus Corynebacterium.

The isopropylmalate synthase may comprise an amino acid sequence of SEQ ID NO: 1 or may consist of the amino acid sequence of SEQ ID NO: 1. Further, if the enzyme has the same or corresponding activity with the isopropylmalate synthase, as the polypeptide having homology of at least 60% or more, 70% or more, 80% or more, 85% or more, 90% or more, 92% or more, 94% or more, 96% or more, 98% or more, or 99% or more, 99.5% or more, or 99.8% or more to the amino acid sequence of SEQ ID NO: 1 regardless of the origin of the microorganism, a polypeptide having an amino acid sequence in which some sequences are deleted, modified, substituted, or added in the amino acid sequence of SEQ ID NO: 1 may also be comprised within the scope of the present application as the isopropylmalate synthase.

That is, even if it is described herein as ‘a polypeptide (or protein) comprising an amino acid sequence represented by a specific sequence number or ‘a polypeptide (or protein) consisting of an amino acid sequence represented by a specific sequence number’, a polypeptide (or protein) having an amino acid sequence in which some sequences are deleted, modified, substituted, or added may also be used as a protein or polypeptide to be mutated in the present application, when it has the same or corresponding activity as a polypeptide (or protein) consisting of the amino acid sequence of the corresponding sequence number. For example, a polypeptide having the same or corresponding activity as the ‘polypeptide consisting of the amino acid sequence of SEQ ID NO: 1’ may belong to the ‘polypeptide consisting of the amino acid sequence of SEQ ID NO: 1’.

The amino acid sequence of the isopropylmalate synthase and the nucleotide sequence of the gene encoding the isopropylmalate synthase may be readily obtained from databases known in the art, such as the National Center for Biotechnology Information (NCBI) and the DNA Data Bank of Japan (DDBJ).

In this specification, a polynucleotide or a polypeptide “comprising a specific nucleic acid sequence (nucleotide sequence) or an amino acid sequence” may mean that the polynucleotide or polypeptide consists of or essentially comprises the specific nucleic acid sequence (nucleotide sequence) or amino acid sequence and may be interpreted as comprising the sequences in which specific nucleic acid sequence or amino acid sequence is mutated (deleted, substituted, modified, and/or added) (or as not excluding the mutation) within the range of maintaining the original function and/or the desired function of the polynucleotide or polypeptide. In one embodiment, a polynucleotide or a polypeptide “comprising a specific nucleic acid sequence (nucleotide sequence) or an amino acid sequence” may mean that the polynucleotide or polypeptide (i) consists of or essentially comprises the specific nucleic acid sequence (nucleotide sequence) or an amino acid sequence, or (ii) consists of or essentially comprises a nucleic acid sequence or an amino acid sequence having a homology of 60% or more, 70% or more, 80% or more, 85% or more, 90% or more, 91% or more, 92% or more, 93% or more, 94% or more, 95% or more, 96% or more, 97% or more, 98% or more, 98% or more, 99.5% or more, or 99.9% or more to a specific nucleic acid sequence (nucleotide sequence) or amino acid sequence, and maintains the original function and/or the desired function.

In this specification, the amino acid sequence of isopropylmalate synthase derived from wild-type Corynebacterium glutamicum (e.g., ATCC13032) was represented by SEQ ID NO: 1, and the nucleotide sequence of the leuA gene encoding the isopropylmalate synthase derived from the wild-type strain was represented by SEQ ID NO: 2. The isopropylmalate synthase was shown to consist of 616 amino acids in SEQ ID NO: 1, but in some literatures, it was known to consist of 581 amino acids as the translation initiation codon is indicated after 35 amino acids, and the amino acid sequence of isopropylmalate synthase consisting of 581 amino acids was represented by SEQ ID NO: 16. The isopropylmalate synthase consisting of 581 amino acids may be comprised within the scope of the present application as the isopropylmalate synthase, and in this case, the position 247 is interpreted as the position 212, which maybe thereby comprised in the scope of the present application.

The mutated polypeptide according to one embodiment may be a polypeptide in which:
an arginine amino acid residue at position 558 (or position corresponding thereto) in the amino acid sequence of SEQ ID NO: 1 is substituted with an amino acid residue other than arginine,
a glycine amino acid residue at position 561 (or position corresponding thereto) in the amino acid sequence of SEQ ID NO: 1 is substituted with an amino acid residue other than glycine, or
the polypeptide is further modified by these two substitutions.

In one embodiment, in the case of isopropylmalate synthase consisting of 581 amino acids of SEQ ID NO: 16, the position 558 is interpreted as the position 523, and the position 561 as the position 526, which is thereby comprised in the scope of the present application.

In this specification, the term “homology” refers to the percentage of identity between two polynucleotides or polypeptide moieties. The homology between sequences from a moiety to another moiety may be determined by the technique known in the art. For example, the homology may be determined by arranging the sequence information and directly arranging the sequence information, i.e., parameters such as score, identity, and similarity, etc., of two polynucleotide molecules or two polypeptide molecules using an easily accessible computer program. The computer program may be BLAST (NCBI), CLC Main Workbench (CLC bio), MegAlign™ (DNASTAR Inc), or the like. Additionally, the homology between polynucleotides may be determined by hybridizing polynucleotides under the condition of forming a stable double-strand between the homologous regions, digesting with a single strand-specific nuclease, followed by confirming size of the digested fragments.

According to one aspect, there may be provided a mutated polypeptide having an isopropylmalate synthase activity in which proline amino acid residue at position 247 (or position corresponding thereto) in the amino acid sequence of SEQ ID NO: 1 is substituted with an amino acid residue other than proline.

According to another aspect, there may be provided a mutated polypeptide having an isopropylmalate synthase activity in which proline amino acid residue at position 212 in the amino acid sequence of SEQ ID NO: 16 is substituted with an amino acid residue other than proline.

In this specification, ‘position ˜ in the amino acid sequence of SEQ ID NO: ˜′ may be used interchangeably with ‘position ˜ from the N-terminal of the polypeptide consisting of (or comprising) the amino acid sequence of SEQ ID NO: ˜.

The amino acids other than proline may comprise arginine, alanine, leucine, isoleucine, valine, phenylalanine, tryptophan, methionine, glycine, serine, threonine, cysteine, tyrosine, asparagine, glutamine, lysine, histidine, aspartic acid and glutamic acid.

According to one embodiment, the mutated polypeptide may be a polypeptide in which proline at position 247 (or a position corresponding thereto) in the amino acid sequence of SEQ ID NO: 1 is substituted with cysteine.

According to one embodiment, the mutated polypeptide may comprise an amino acid sequence of SEQ ID NO: 3 or may consist of the amino acid sequence of SEQ ID NO: 3.

The mutated polypeptide according to one embodiment may be a polypeptide in which the amino acid residue at a position corresponding to the position 247 (in the case of SEQ ID NO: 16, the position 212) in the amino acid sequence of a polypeptide having homology of 60% or more, 70% or more, 80% or more, 85% or more, 90% or more, 92% or more, 94% or more, 96% or more, 98% or more, or 99% or more, 99.5% or more, or 99.8% or more to the amino acid sequence of SEQ ID NO: 1 (or SEQ ID NO: 16) is substituted with another amino acid residue.

The mutated polypeptide according to one embodiment may have increased L-leucine production activity than the polypeptide comprising the amino acid sequence of SEQ ID NO: 1 (or SEQ ID NO: 16) by substituting proline at position 247 in the amino acid sequence of SEQ ID NO: 1 (or proline at position 212 in the amino acid sequence of SEQ ID NO: 16) or the amino acid at a position corresponding thereto with another amino acid.

According to one embodiment, the mutated polypeptide, in which a proline amino acid residue at position 247 or an amino acid residue at a position corresponding thereto in the amino acid sequence of the polypeptide having the homology of 70% or more, 80% or more, 85% or more, 90% or more, 92% or more, 94% or more, 96% or more, 98% or more, or 99% or more to the amino acid sequence of SEQ ID NO: 1 or the amino acid sequence of SEQ ID NO: 1 is substituted with another amino acid residue, may have an isopropylmalate synthase activity. Said ‘another amino acid residue’ may mean other type of amino acid residue other than the amino acid (e.g., proline) that was present at said position (position 247 in the amino acid sequence of SEQ ID NO: 1 or position corresponding thereto) before being substituted.

The mutated polypeptide according to one embodiment may have increased L-leucine production activity than the isopropylmalate synthase derived from a wild-type strain (e.g., isopropylmalate synthase comprising (or consisting of) the amino acid sequence of SEQ ID NO: 1 or SEQ ID NO: 16) by substituting proline at position 247 (or position 212) in the amino acid sequence of SEQ ID NO: 1 (or SEQ ID NO: 16) or the amino acid residue at a position corresponding thereto, with another residue.

The mutated polypeptide according to one embodiment may have increased enzymatic activity of isopropylmalate synthase than the isopropylmalate synthase derived from a wild-type strain (e.g., isopropylmalate synthase consisting of the amino acid sequence of SEQ ID NO: 1 (or SEQ ID NO: 16)) by substituting proline at position 247 (or position 212) in the amino acid sequence of SEQ ID NO: 1 (or SEQ ID NO: 16) or the amino acid residue at a position corresponding thereto, with another residue.

In this specification, the term “increase in activity of isopropylmalate synthase” refers to an increase in the conversion activity to isopropylmalate. Therefore, the mutated polypeptide according to one embodiment may have a higher level of the isopropylmalate conversion activity compared to a polypeptide having an isopropylmalate synthase activity comprising the amino acid sequence of SEQ ID NO: 1 (or SEQ ID NO: 16).

In this specification, the term “increase in activity” may be used interchangeably with “enhanced activity”. Further, isopropylmalate is one of the precursors of L-leucine, and thus, using the mutated polypeptide according to one embodiment results in producing a higher level of L-leucine compared to a polypeptide having an isopropylmalate synthase activity comprising the amino acid sequence of SEQ ID NO: 1 (or SEQ ID NO: 16).

The isopropylmalate conversion activity may be directly confirmed by measuring the level of isopropylmalate produced, or may be indirectly confirmed by measuring the level of CoA produced. The enzymatic activity of isopropylmalate synthase may be measured by a known method, for example, may be measured according to the method described in Kohlhaw et al. (Methods in Enzymology 166:423-9 (1988)) and the change in absorbance at 412 nm due to thionitrobenzoate (TNB) formed from DTNB (5,5′-dithiobis-(2-nitrobenzoic acid), Ellman's reagent) by reduction using the CoA produced may be measured, thereby determining the activity of the isopropylmalate synthase enzyme.

The mutated polypeptide according to one embodiment may reduce feedback inhibition by L-leucine and/or a derivative thereof as compared with isopropylmalate synthase derived from a wild-type strain (for example isopropylmalate synthase consisting of the amino acid sequence of SEQ ID NO: 1) by substituting proline at position 247 (or position 212) in the amino acid sequence of SEQ ID NO: 1 (or SEQ ID NO: 16) or the amino acid sequence at a position corresponding thereto with another amino acid.

In this specification, the term “feedback inhibition” means that the end product of the enzyme system inhibits a reaction at the initial stage of the enzyme system. For example, the feedback inhibition may mean that L-leucine or a derivative thereof inhibits the activity of isopropylmalate synthase, which mediates the first step of the biosynthetic pathway thereof. Therefore, when the feedback inhibition of isopropylmalate synthase is reduced (or released), the productivity of L-leucine may be increased compared to the case where it is not.

In this specification, the term “derivative” may refer to compounds which are known to be capable of inhibiting an ability to produce L-leucine from microorganisms by inducing feedback inhibition in relation to the biosynthesis of L-leucine which is the final product of the present invention. Examples thereof may comprise isoleucine, terleucine, norleucine, and/or cycloleucine, etc.

According to one embodiment, the mutated polypeptide may be a polypeptide in which a position other than position 247 (or position 212) in the amino acid sequence of SEQ ID NO: 1 (or SEQ ID NO: 16) is additionally mutated, thereby synergistically increasing one or more effects selected from the group consisting of (1) an increase in L-leucine production activity; (2) an increase in enzymatic activity of isopropylmalate synthase; and (3) a reduction of feedback inhibition by L-leucine and/or a derivative thereof.

According to one embodiment, the mutated polypeptide may further comprise mutations that may exhibit one or more effects selected from the group consisting of (1) an increase in L-leucine production activity; (2) an increase in enzymatic activity of isopropylmalate synthase; and (3) a reduction of feedback inhibition by L-leucine and/or a derivative thereof.

The mutated polypeptide may be a polypeptide in which:
an arginine amino acid residue at position 558 (or a position corresponding thereto) in the amino acid sequence of SEQ ID NO: 1 is substituted with an amino acid residue other than arginine,
a glycine amino acid residue at position 561 (or a position corresponding thereto) in the amino acid sequence of SEQ ID NO: 1 is substituted with an amino acid residue other than glycine, or the polypeptide is further modified by these two substitutions.

The mutated polypeptide may be a polypeptide in which:
an arginine amino acid residue at position 523 (or a position corresponding thereto) in the amino acid sequence of SEQ ID NO: 16 is substituted with an amino acid residue other than arginine,
a glycine amino acid residue at position 526 (or a position corresponding thereto) in the amino acid sequence of SEQ ID NO: 16 is substituted with an amino acid residue other than glycine, or the polypeptide is further modified by these two substitutions.

The amino acid other than arginine may comprise alanine, leucine, isoleucine, valine, proline, phenylalanine, tryptophan, methionine, glycine, serine, threonine, cysteine, tyrosine, asparagine, glutamine, lysine, histidine, aspartic acid, and glutamic acid; and the amino acid other than glycine may comprise alanine, leucine, isoleucine, valine, proline, phenylalanine, tryptophan, methionine, arginine, serine, threonine, cysteine, tyrosine, asparagine, glutamine, lysine, histidine, aspartic acid, and glutamic acid; but not limited thereto.

According to one embodiment, the amino acid other than arginine may be histidine.

According to one embodiment, the amino acid other than glycine may be aspartic acid.

According to one embodiment, the mutated polypeptide may be a polypeptide in which:
an arginine amino acid residue at position 558 in the amino acid sequence of SEQ ID NO: 1 is substituted with a histidine amino acid residue,
a glycine amino acid residue at position 561 in the amino acid sequence of SEQ ID NO: 1 is substituted with an aspartic acid amino acid residue, or
the polypeptide is further modified by these two substitutions.

According to one embodiment, the mutated polypeptide may comprise the amino acid sequence of SEQ ID NO: 5, or may consist of the amino acid sequence of SEQ ID NO: 5.

In the mutated polypeptide in which amino acid residue at position 247 (or position 212) in the amino acid sequence of SEQ ID NO: 1 (or SEQ ID NO: 16) is substituted with an amino acid residue other than proline,
a mutated polypeptide in which arginine amino acid residue at position 558 (or position 523) in the amino acid sequence of SEQ ID NO: 1 (or SEQ ID NO: 16) is substituted with an amino acid residue other than arginine, the glycine amino acid residue at position 561 (or position 526) is substituted with an amino acid residue other than glycine, or the polypeptide is further modified by these two substitutions, may be a polypeptide in which:
as compared with a mutated polypeptide in which (i) isopropylmalate synthase derived from a wild-type strain (for example, isopropylmalate synthase comprising the amino acid sequence of SEQ ID NO: 1 or SEQ ID NO: 16) or (ii) the proline amino acid residue at position 247 (or positon 212) in the amino acid sequence of SEQ ID NO: 1 (or SEQ ID NO: 16) is substituted with an amino acid residue other than proline,
one or more effects selected from the group consisting of the following (1) to (3) may be increased:
(1) increase in L-leucine production activity;
(2) increase in enzymatic activity of isopropylmalate synthase; and
(3) reduction of feedback inhibition by L-leucine and/or a derivative thereof.

According to one embodiment, depending on the combination of mutations in which amino acid at positions 247, 558, and/or 561 (or positions 212, 523, and/or 526) in the amino acid sequence of SEQ ID NO: 1 (or SEQ ID NO: 16) is substituted with another type of amino acid, the mutated polypeptide according to one embodiment may have synergistically increased one or more effects selected from the group consisting of the following (1) to (3):
(1) increase in L-leucine production activity;
(2) increase in enzymatic activity of isopropylmalate synthase; and
(3) reduction of feedback inhibition by L-leucine and/or a derivative thereof.

The effects of (1) to (3) above are the same as described above.

According to another aspect, there may be provided a polynucleotide encoding the mutated polypeptide. In this specification, the term “polynucleotide” is a polymer of nucleotides in which nucleotide monomers are covalently linked in a long chain shape, and is a DNA or RNA strand having a certain length or longer. More specifically, it may mean a polynucleotide fragment encoding the mutated polypeptide.

The mutated polypeptide is the same as described above.

The polynucleotide may comprise, without limitation, a polynucleotide sequence encoding the mutated polypeptide of the present application.

According to one embodiment, it may be a polynucleotide encoding a polypeptide in which a proline amino acid residue at position 247 (or position 212) in the amino acid sequence of SEQ ID NO: 1 (or SEQ ID NO: 16) is substituted with an amino acid residue other than proline, or a polynucleotide encoding a polypeptide which has a homology of 60% or more, 70% or more, 80% or more, 85% or more, 90% or more, 92% or more, 94% or more, 96% or more, 98% or more, or 99% or more, 99.5% or more, or 99.8% or more to the amino acid sequence of SEQ ID NO: 1 (or SEQ ID NO: 16), in which an amino acid residue at a position corresponding to position 247 (or position 212) in the amino acid sequence of SEQ ID NO: 1 (or SEQ ID NO: 16) is substituted with other amino acid residue, or encoding a polypeptide in which some of the sequences are deleted, modified, substituted or added thereof.

According to one embodiment, the polynucleotide may comprise without limitation as long as it is a probe that may be prepared from a known gene sequence, for example, a probe capable of hybridizing with a complementary sequence for all or part of the polynucleotide sequence under stringent conditions, and comprising a sequence corresponding to a sequence encoding a protein variant in which an amino acid at position 247 (or position 212) or a corresponding thereto in the amino acid sequence of SEQ ID NO: 1 (or SEQ ID NO: 16) is substituted with other amino acid. The term “stringent conditions” refers to conditions under which a so-called specific hybrid is formed while non-specific hybrids are not formed. Examples of such conditions comprise conditions under which genes having high degrees of homology, such as genes having a homology of 60% or more, 70% or more, 80% or more, 85% or more, 90% or more, 92% or more, 94% or more, 96% or more, 98% or more, or 99% or more, 99.5% or more, or 99.8% or more, hybridize with each other while genes having low degrees of a homology do not hybridize with each other, or conditions under which genes are washed 1 time, and specifically 2 and 3 times, at a temperature and a salt concentration equivalent to 60° C., 1×SSC, and 0.1% SDS, specifically 60° C., 0.1×SSC, and 0.1% SDS, and more specifically 68° C., 0.1×SSC, and 0.1% SDS, which are the conditions for washing of ordinary Southern hybridization (Sambrook et al., Molecular Cloning: A Laboratory Manual, 3rd Ed., Cold Spring Harbor Laboratory Press, Cold Spring Harbor, N.Y. (2001)).

The probe used in the hybridization may be a part of the complementary sequence of the nucleotide sequence. Such probe may be constructed by PCR using an oligonucleotide prepared based on a known sequence as a primer and using a gene fragment containing such nucleotide sequence as a template. For example, a gene fragment having a length of about 300 bp may be used as a probe. More specifically, in the case of using a probe having a length of about 300 bp, 50° C., 2×SSC, and 0.1% SDS may be suggested for the washing conditions of hybridization.

According to one embodiment, the polynucleotide may comprise the nucleotide sequence of SEQ ID NO: 4 or SEQ ID NO: 6, or may consist of the nucleotide sequence of SEQ ID NO: 4 or SEQ ID NO: 6, and the polynucleotide that may be translated into the mutated polypeptide according to one embodiment by codon degeneracy may also be comprised in the scope of the present application.

According to another aspect, there may be provided a vector comprising the polynucleotide.

In this specification, the term “vector” refers to any carrier for cloning and/or transferring nucleotides to a host cell. A vector may be a replicon to allow for the replication of the fragments combined with other DNA fragments. “Replicon” refers to any genetic (e.g., plasmid, phage, cosmid, chromosome, and virus) unit acting as a self-replicating of DNA replication in vivo, that is, replicable by self-regulation.

In one embodiment, the vector may be a DNA construct comprising a polynucleotide sequence encoding the target protein, which is operably linked to a suitable regulatory sequence such that the target protein may be expressed in an appropriate host. The regulatory sequence may comprise a promoter capable of initiating transcription, any operator sequence for the control of the transcription, a sequence encoding an appropriate mRNA ribosome-binding domain, and a sequence controlling the termination of transcription and translation. After being transformed into a suitable host cell, the vector may be replicated or function irrespective of the host genome, and it may be integrated into the host genome itself.

In this specification, the term “operably linked” means that the gene sequence is functionally linked to a promoter sequence which initiates and mediates the transcription of the polynucleotide encoding the polypeptide.

The vector is not particularly limited as long as it is capable of replicating in a host cell, and any vector known in the art may be used. For example, the vector may be natural or recombinant plasmids, cosmids, viruses, and bacteriophages. As a phage vector or a cosmid vector, MBL3, MBL4, IXII, ASHII, APII, t10, t11, pWE15, M13, λEMBL3, λEMBL4, λFIXII, λDASHII, λZAPII, λgt10, λgt11, Charon4A, and/or Charon21A, and the like may be used, and as a plasmid vector, pDZ vectors, pBR based, pUC based, pBluescript II based, pGEM based, pTZ based, pCL based, and/or pET based vectors, and the like may be used. For example, pCR2.1, pDC, pDZ, pACYC177, pACYC184, pCL, pECCG117, pUC19, pBR322, pMW118 and/or pCC1BAC vectors and the like may be used.

According to another aspect, there may be provided a microorganism (or recombinant cell) comprising at least one selected from the group consisting of the mutated polypeptide, the polynucleotide encoding the mutated polypeptide, and the vector comprising the polynucleotide.

The details concerning the mutated polypeptide, the polynucleotide and the vector are the same as described above.

The microorganism (or recombinant cell) may further comprise a mutation that increases L-leucine production, and the position of the mutation and/or the type of the gene to be mutated may be comprised without limitation as long as it increases L-leucine production.

The microorganism (or recombinant cell) may further comprise a mutation that enhances the activity of an enzyme involved in L-leucine biosynthesis. Enhancing the activity of the enzyme is the same as described above.

The microorganism (recombinant cell) may be used without limitation as long as it is a cell capable of transformation.

The microorganism (or recombinant cell) may have an L-leucine producing ability. The microorganism (or recombinant cell) may have a more improved L-leucine-producing ability or may have an L-leucine-producing ability that a cell, a parent strain, and/or a wild-type strain prior to recombination do not have.

The microorganism (or recombinant cell) may be a polypeptide in which an amino acid sequence corresponding to isopropylmalate synthase is mutated, so that its activity is increased compared to a wild-type strain, a parent strain, and/or a cell prior to recombination, or feedback inhibition for L-leucine and its derivative is released, or both the increase in the activity of the enzyme and the release of the feedback inhibition may be achieved.

The microorganism (or recombinant cell) may be one in which a polynucleotide according to one embodiment is integrated into a chromosome, and for example, the polynucleotide according to one embodiment may be exchanged for a native leuA gene (gene encoding isopropylmalate synthase) at a gene site in a chromosome or integrated into an additional gene site.

In this specification, “having an L-leucine producing ability” refers to microorganisms in which leucine producing ability is imparted to cells and/or microorganisms that do not have leucine producing ability, or cells and/or microorganisms that have naturally leucine-producing ability. For example, microorganisms of the genus Corynebacterium having an L-leucine producing ability refers to a microorganism of the genus Corynebacterium that has an improved L-leucine producing ability by inserting an external gene related to the mechanism of leucine production, or enhancing or inactivating the activity of a native gene or a natural microorganism itself. The microorganism may be a microorganism of the genus Corynebacterium.

The microorganism of the genus Corynebacterium may be Corynebacterium glutamicum, Corynebacterium ammoniagenes, Corynebacterium acetoacidophilum, Corynebacterium acetoglutamicum, Corynebacterium alkanolyticum, Corynebacterium callunae, Corynebacterium lilium, Corynebacterium melassecola, Corynebacterium thermoaminogenes, Corynebacterium efficiens, and/or Corynebacterium herculis, etc.

The microorganism (or recombinant cell) may comprise being artificially produced through transformation and/or occurred naturally. For example, the microorganism (or recombinant cell) may be transformed with a polynucleotide encoding a mutated polypeptide according to one embodiment or a vector comprising the same.

In this specification, the term “transformation” refers to the introduction of a gene or a polynucleotide into a host cell so that it may be expressed in a host cell, and the transformed gene or polynucleotide may comprise both a form inserted into the chromosome of a host cell and a form located outside the chromosome, without limitation, as long as it may be expressed in the host cell.

In this specification, the method of transformation comprises without limitation as long as it is a method of introducing a gene into a cell, and may be carried out by selecting a suitable standard technique as known in the art, depending on the host cell. For example, electroporation, calcium phosphate (CaPO₄) precipitation, calcium chloride (CaCl₂) precipitation, microinjection, retroviral infection, polyethylene glycol (PEG) method, DEAE-dextran method, cationic liposome method, and/or lithium acetate-DMSO method, and the like may be used, but are not limited thereto.

The microorganism (or recombinant cell) may be one in which a polynucleotide sequence according to one embodiment is integrated into a chromosome. The homologous recombination allows the exchange of DNA fragments on the chromosome for a polynucleotide according to one embodiment that is transferred by the vector into a cell, along with the use of a vector according to one embodiment. For efficient recombination between the cyclic DNA molecule of the vector and the target DNA on the chromosome, the exchanged DNA region comprising the polynucleotide according to one embodiment is provided with a nucleotide sequence homologous to the target site at the end; and these determine a vector integration site and a DNA exchange site. For example, a polynucleotide according to one embodiment may be exchanged for a native leuA gene at a native gene site within a chromosome or may be incorporated into an additional gene site.

According to another aspect, there may be provided a method for producing L-leucine, which comprises the step of culturing the microorganism (or recombinant cell) in a medium.

In one embodiment, the method may further comprise the step of collecting L-leucine from the cultured medium or microorganism (or recombinant cell).

The culturing may be carried out according to an appropriate medium and culture conditions known in the art, must meet the requirements of a specific strain in an appropriate manner, and may be appropriately modified by a person skilled in the art.

The culture method may comprise, for example, batch culture, continuous culture, fed-batch culture, or a combination culture thereof, but is not limited thereto.

The medium for culturing a microorganism (or a recombinant cell) may refer to known literature (Manual of Methods for General Bacteriology. American Society for Bacteriology. Washington D.C., USA, 1981), but is not limited thereto.

According to one embodiment, the medium may comprise various carbon sources, nitrogen sources, and trace element components, and the recombinant cells may be cultured while adjusting the temperature and/or pH in an ordinary medium comprising an appropriate carbon source, nitrogen source, amino acid, vitamin, and the like. The carbon source comprises sugars and carbohydrates such as glucose, sucrose, lactose, fructose, maltose, starch, and/or cellulose, oils and fats such as soybean oil, sunflower oil, castor oil, and/or coconut oil, fatty acids such as palmitic acid, stearic acid, and/or linoleic acid, alcohols such as glycerol, and/or ethanol, organic acids such as acetic acid. These materials may be used alone or in combination of two or more, but are not limited thereto. The nitrogen source that may be used may comprise peptone, yeast extract, meat juice, malt extract, corn steep liquor, soybean flour and urea or inorganic compounds, for example, ammonium sulfate, ammonium chloride, ammonium phosphate, ammonium carbonate and ammonium nitrate. The nitrogen source may also be used alone or in combination of two or more, but is not limited thereto. A phosphorus source that may be used may comprise potassium dihydrogen phosphate or dipotassium hydrogen phosphate or corresponding sodium-containing salts, but is not limited thereto. Further, the medium may comprise a metal salt such as magnesium sulfate or iron sulfate required for growth, but is not limited thereto. In addition, essential growth substances such as amino acids and vitamins may be comprised. Further, precursors suitable for the medium may be used. The medium or individual components may be added in a batch or continuous manner to the culture medium during the culture process, but is not limited thereto.

According to one embodiment, compounds such as ammonium hydroxide, potassium hydroxide, ammonia, phosphoric acid, and sulfuric acid may be added to a culture medium of microorganism in an appropriate manner during culture to adjust the pH of the culture medium. In addition, the formation of bubbles may be suppressed by using an antifoaming agent such as fatty acid polyglycol ester during culture. Additionally, in order to maintain the aerobic state of the culture medium, oxygen or an oxygen-containing gas (e.g., air) may be injected into the culture medium. The temperature of the culture medium may be 20° C. to 45° C., 25° C. to 40° C., or 30° C. to 37° C. The culture period may be continued until a useful substance (e.g., L-leucine) is obtained in a required production amount, and may be, for example, 10 to 160 hours.

The step of isolating or collecting L-leucine from the cultured microorganism (or recombinant cell) and/or cultured medium may be carried out using a suitable method known in the art depending on the culturing method. For example, centrifugation, filtration, extraction, spraying, drying, distillation, precipitation, crystallization, electrophoresis, fractional dissolution (e.g., ammonium sulfate precipitation), and/or chromatography (e.g., ion exchange, affinity, hydrophobicity and size exclusion) may be used, but is not limited thereto. The cultured medium refers to a medium in which a microorganism (or a recombinant cell) was cultured.

According to one embodiment, the step of isolating or collecting L-leucine maybe carried out by centrifuging the culture at low speed to remove the biomass, and isolating the resulting supernatant via ion exchange chromatography.

The method for producing L-leucine may further comprise a step of purifying L-leucine.

According to another aspect, there may be provided a composition for producing L-leucine comprising at least one selected from the group consisting of a mutated polypeptide of the present application, a polynucleotide encoding the mutated polypeptide, a vector comprising the polynucleotide, and the microorganism (for example, a mutated polypeptide of the present application, a polynucleotide of the present application, and/or a microorganism (recombinant cell) comprising a vector of the present application).

A composition of the present may further comprise any suitable excipient commonly used in compositions for the production of amino acids, and the excipient, for example, may be preservative, a wetting agent, a dispersing agent, a suspending agent, a buffering agent, a stabilizing agent, or an isotonic agent, but is not limited thereto.

In the composition of the present application, mutated polypeptides, polynucleotides, vectors, microorganisms, and media are the same as those described in the other aspects above.

According to another aspect, there may be provided a use of at least one selected from the group consisting of the mutated polypeptide, a polynucleotide encoding the mutated polypeptide, a vector comprising the polynucleotide, and the microorganism (for example, a mutated polypeptide of the present application, a polynucleotide of the present application, and/or a microorganism (recombinant cell) comprising a vector of the present application) for producing L-leucine.

In the use of the present application, mutated polypeptides, polynucleotides, vectors, microorganisms, and media are the same as those described in the other aspects above.

### Advantageous Effects

By using the mutated polypeptide according to one embodiment, it is possible to produce L-leucine in high yield.

### DETAILED DESCRIPTION OF THE EMBODIMENTS

Hereinafter, the present invention will be described in more detail with reference to the following examples, but the scope of the present invention is not intended to be limited by these examples.

### Example 1. Construction of DNA Library Encoding Mutated Isopropylmalate Synthase

### Example 1-1. Construction of Vector Comprising leuA

In order to construct a leuA mutant library encoding a variant having an isopropylmalate synthase activity, a recombinant vector comprising leuA was first constructed.

In order to amplify the leuA gene (SEQ ID NO: 2) encoding the LeuA (2-isopropylmalate synthase) protein (SEQ ID NO: 1, Uniprot accession code: P42455) derived from wild-type Corynebacterium glutamicum, the PCR method was performed using the chromosome of Corynebacterium glutamicum ATCC13032 wild strain as a template and using primers of SEQ ID NOs: 7 and 8 under the following conditions repeated 25 cycles: denaturation at 94° C. for 1 min, annealing at 58° C. for 30 sec, and polymerization at 72° C. for 1 min using Pfu DNA polymerase. The specific sequences of the primers used are shown in Table 1 below.

The amplified PCR product was cloned into E. coli vector pCR2.1 using the TOPO Cloning Kit (Invitrogen) according to the manufacturer's manual to obtain ‘pCR-leuA’.

TABLE 1

| SEQ ID NO | Sequence (5′→3′) |
|---|---|
| SEQ ID NO: 7 | CTAATCTCGAGGTCACCCATGTCTCCTAAC |
| SEQ ID NO: 8 | GGCTGGCGGCGTTTAAAACCGGTTGAT |

### Example 1-2. Construction of leuA Mutant Library

Based on the vector constructed in Example 1-1, a leuA mutant library was constructed using an error-prone PCR kit (clontech Diversify® PCR Random Mutagenesis Kit). Under conditions where 0 to 3 mutations occurred per 1000 bp, a PCR reaction was performed using the pCR-leuA vector as a template and using primers of SEQ ID NO: 7 and SEQ ID NO: 8. Specifically, as the condition where 0 to 3 mutations occurred per 1000 bp, the PCR reaction was performed under the following conditions: pre-heating at 94C for 30 sec, and then 25 cycles of 94° C. for 30 sec and 68° C. for 1 min and 30 sec. The obtained PCR product was used as a megaprimer (50˜125 ng), and the PCR reaction was performed by repeating 25 cycles of 95° C. for 50 sec, 60° C. for 50 sec, and 68° C. for 12 m, followed by DpnI-treatment. The DpnI-treated PCR product was transformed into E. coli DH5a through a heat shock method and spread on LB solid medium containing kanamycin (25 mg/L). After selecting 20 transformed colonies, plasmids were obtained, and the nucleotide sequence was analyzed. As a result, it was confirmed that mutations were introduced at mutually different positions with a frequency of 2mutations/kb. About 20,000 transformed E. coli colonies were taken, and plasmids were extracted, which was named ‘pTOPO-leuA-library’.

### Example 2. Evaluation of Constructed Library and Selection of Variants

### Example 2-1. Selection of Mutant Strains with Increased L-Leucine Production Amount

The pTOPO-leuA-library constructed in Example 1-2 was transformed into wild-type Corynebacterium glutamicum ATCC13032 by electroporation therapy, the transformed strain was spread on a nutrient medium (Table 2) containing 25 mg/L kanamycin, and colonies of 10,000 strains into which the mutant gene was inserted were selected. Each selected colony was named ATCC13032/pTOPO_leuA (mt) 1 to ATCC13032/pTOPO_leuA (mt) 10,000. To identify colonies with increased L-leucine production amount among the 10,000 colonies obtained, the fermentation titer was evaluated for each colony in the following manner.

TABLE 2

| Type of | |
|---|---|
| medium | Components |
| Production | Glucose 100 g, (NH₄)₂SO₄ 40 g, soy protein 2.5 g, corn |
| medium | steep solids 5 g, urea 3 g, KH₂PO₄ 1 g, MgSO₄•H₂O |
| | 0.5 g, biotin 100 μg, thiamine hydrochloride 1,000 μg, |
| | calcium-pantothenate 2000 μg, nicotinamide 3,000 μg, |
| | CaCO₃ 30 g; (based on 1 liter of distilled water), pH 7.0 |
| Nutrient | Glucose 10 g, meat juice 5 g, polypeptone 10 g, sodium |
| medium | chloride 2.5 g, yeast extract 5 g, agar 20 g, urea 2 g |
| | (based on 1 liter of distilled water) |

Each colony was inoculated into a 250 ml corner-baffled flask containing 25 μg/ml kanamycin in 25 ml of autoclave-sterilized production medium (Table 2) using a platinum loop, and then subjected to shaking culture at 30° C. with a shaking rate of 200 rpm for 60 hours. After completion of the culture, L-leucine production amount was measured by a method using high performance liquid chromatography (HPLC, SHIMAZDU LC20A).

Of the 10,000 colonies obtained, one strain (ATCC13032/pTOPO_leuA (mt) 5306) having the most improved L-leucine producing ability compared to the wild-type Corynebacterium glutamicum strain (ATCC13032) was selected. The concentration of L-leucine produced in the selected strain (ATCC13032/pTOPO_leuA (mt) 5306) is shown in Table 3 below.

TABLE 3

| Strain name | L-leucine (g/L) |
|---|---|
| ATCC13032 | 0.87 |
| ATCC13032/pTOPO_leuA(mt)5306 | 1.32 |

As shown in Table 3, Corynebacterium glutamicum ATCC13032/pTOPO_leuA (mt) 5306 strain having a mutation in the leuA gene increased an L-leucine producing ability by about 1.5 times as compared with the parent strain, Corynebacterium glutamicum ATCC13032.

### Example 2-2. Confirmation of Mutations in Mutant Strains with Increased L-Leucine Production

In order to confirm the leuA gene mutation of Corynebacterium glutamicum ATCC13032/pTOPO_leuA (mt) 5306 strain, PCR was performed using the DNA of ATCC13032/pTOPO_leuA (mt) 5306 strain as a template and using primers of SEQ ID NO: 9 and SEQ ID NO: 10 listed in Table 4 under the following conditions: denaturation at 94° C. for 5 min, then 30 cycles of 94° C. for 30 sec, 55° C. for 30 sec, and 72° C. for 1 min and 30 sec, followed by 72° C. for 5 min, and DNA sequencing was performed.

TABLE 4

| SEQ ID NO | Sequence (5′→3′) |
|---|---|
| SEQ ID NO: 9 | AACACGACCGGCATCCCGTCGC |
| SEQ ID NO: 10 | AAATCATTTGAGAAAACTCGAGG |

As a result of the sequencing, it was confirmed that in the ATCC13032/pTOPO_leuA (mt) 5306 strain, CC, which is the 739th and 740th nucleotides of the leuA gene, is substituted with TG. This means capable of encoding a variant (hereinafter, P247C) in which proline, which is an amino acid at position 247 (position 212 if the LeuA protein consist 581 amino acids (SEQ ID NO: 16) as the translation initiation codon is indicated after 35 amino acids, based on the known literature; hereinafter, represented only by position 247) of the LeuA protein is substituted with cysteine. The amino acid sequence of the LeuA variant (P247C) and the nucleotide sequence of the leuA variant encoding the same are the same as those of SEQ ID NO: 3 and SEQ ID NO: 4, respectively.

In the following examples, it was attempted to confirm whether the mutation (P247C) affects the L-leucine production amount of the microorganism of the genus Corynebacterium.

### Example 3. Confirmation of L-Leucine Producing Ability of Selected Mutant Strains

### Example 3-1. Construction of Insertion Vectors Containing leuA Mutation

In this example, in order to introduce the selected mutation (P247C) into strains using the site directed mutagenesis method, it was attempted to construct a vector for insertion. PCR was performed using a chromosome of Corynebacterium glutamicum wild type (ATCC13032) as a template and using primers of SEQ ID NOs: 11 and 12 and primer pairs of SEQ ID NOs: 13 and 14. Specifically, PCR was performed under the following conditions: denaturation at 94° C. for 5 min, then 30 cycles of 94° C. for 30 sec, 55° C. for 30 sec, and 72° C. for 1 min and 30 sec, followed by 72° C. for 5 min. The specific sequences of the primers used are shown in Table 5.

TABLE 51

| SEQ ID NO | Sequence (5′→3′) |
|---|---|
| SEQ ID NO: | GTGAATTCGAGCTCGGTACCCAAATCATTTGAGAAAACT |
| 11 | CGAGGC |
| SEQ ID NO: | GGTGATCATCTCAACGGTGGAACACAGGTTGATGATCAT |
| 12 | TGGGTT |
| SEQ ID NO: | AACCCAATGATCATCAACCTGTGTTCCACCGTTGAGATG |
| 13 | ATCACC |
| SEQ ID NO: | GGTCGACTCTAGAGGATCCCCAAGAAGGCAACATCGGAC |
| 14 | AGC |

The resulting PCR product was cleaved with Smal restriction enzyme to obtain a linear pDZ vector, and the homologous sequence of the terminal 15 base between DNA fragment was fused and cloned using an In-Fusion enzyme, thereby a vector ‘pDZ-leuA (P247C)’ in which proline (Pro), which is the 247th amino acid of LeuA, was substituted with cysteine (Cys) was constructed.

### Example 3-2. Introduction of leuA Gene Mutation in ATCC13032 Strain

The pDZ-leuA (P247C) vector constructed in Example 3-1 was transformed into ATCC13032 by electroporation therapy, and strains having a vector inserted on a chromosome by recombination of the homologous sequence were selected in a medium containing 25 mg/L of kanamycin. The selected primary strain was again subjected to a secondary cross-over, and a strain in which a mutation was introduced into the leuA gene was selected. Whether or not the leuA gene mutation was introduced into the finally transformed strain was confirmed through nucleotide sequence analysis after PCR was performed using the primers of SEQ ID NO: 9 and SEQ ID NO: 15 under the following conditions: 94° C. for 5 min, then 30 cycles of 94° C. for 30 sec/55° C. for 30 sec/72° C. for 90 sec, followed by 72° C. for 5 min. As a result of nucleotide sequence analysis, it was confirmed that CC, in which the 739th and 740th nucleotides of the leuA gene in strain chromosome were substituted with TG, and a leuA mutation encoding LeuA, in which the 247th amino acid proline (Pro) was substituted with cysteine (Cys), was introduced in the strain. The produced strain was named ‘ATCC13032_leuA_P247C’. The specific sequences of the primers used are shown in Tables 4 and 6

TABLE 61

| SEQ ID NO | Sequence (5′→3′) |
|---|---|
| SEQ ID NO: 15 | ATCCATTCAATGGAGTCTGCG |

### Example 3-3. Evaluation of L-Leucine Producing Ability of Mutant Strains

In order to evaluate the L-leucine producing ability of the ATCC13032 leuA P247C strain produced in Example 3-2, flask fermentation titer was evaluated similarly to the method of Example 2. The parent strains, Corynebacterium glutamicum ATCC13032 and ATCC13032_leuA_P247C, were respectively inoculated into a 250 ml corner-baffled flask containing 25 ml of production medium using a platinum loop, and then subjected to shaking culture at 30° C. with a shaking rate of 200 rpm for 60 hours for producing L-leucine. After completion of the culture, L-leucine production amount was measured by HPLC, and the concentration of L-leucine in the culture medium for each strain is shown in Table 7 below.

TABLE 7

| Strain name | L-leucine (g/L) |
|---|---|
| ATCC13032 | 0.87 |
| ATCC13032_leuA_P247C | 1.35 |

As shown in Table 7, ATCC13032_leuA_P247C improved the yield of L-leucine by about 1.55 times as compared with the parent strain, Corynebacterium glutamicum ATCC13032.

### Example 4. Evaluation of Leucine-Producing Ability in Leucine-Producing Mutant Strains

Since the wild-type strain of the genus Corynebacterium produces a trace amount of L-leucine, a leucine-producing strain derived from ATCC13032 was produced, and the mutation (P247C) selected in Example 2 was introduced to confirm an L-leucine-producing ability. The specific experiment was performed as follows.

### Example 4-1. Production of L-Leucine-Producing Strain (CJL-8100 Strain)

As a strain for the production of high concentration L-leucine, a strain derived from ATCC13032 comprising as follow mutations was produced: (1) a mutation (R558H) in which arginine, which is the 558th amino acid of the LeuA protein, was substituted with histidine, by substituting G, which is the 1673th nucleotide of the leuA gene, with A, and (2) a mutation (G561D) in which glycine, which is the 561th amino acid, was substituted with aspartic acid, by substituting GC, which is the 1682th and 1683th nucleotides of the leuA gene, with AT.

Specifically, the pDZ-leuA (R558H, G561D) vector comprising the leuA gene mutation (KR10-2018-0077008 A) was transformed into Corynebacterium glutamicum ATCC13032 by electroporation therapy, and a strain having a vector inserted on a chromosome by recombination of the homologous sequence was selected in a medium containing 25 mg/L of kanamycin. The selected primary strain was again subjected to a secondary cross-over, and a strain into which the mutation of the leuA gene was introduced was selected. Finally, whether or not to introduce mutations of the transformed strain was then confirmed, in which PCR was performed using the primers of SEQ ID NO: 7 and SEQ ID NO: 13 under the following conditions: 94° C. for 5 min, then 30 cycles of 94° C. for 30 sec/55° C. for 30 sec/72° C. for 90 seconds, followed by 72° C. for 5 min, and the nucleotide sequence was analyzed, thereby confirming that the R558H and G561D mutations were introduced. The specific sequences of the primers used are listed in Tables 1 and 5. The ATCC13032_leuA_(R558H, G561D) strain transformed with the pDZ-leuA (R558H, G561D) vector was named “CJL-8100”.

### Example 4-2. Construction of Insertion Vector Comprising leuA Mutation

In this example, in order to introduce the mutation (P247C) selected in Example 2 into CJL-8100, which is an L-leucine-producing strain in which two mutations (R558H, G561D) were introduced into LeuA, it was attempted to construct a vector for insertion.

PCR was performed using the chromosome of the CJL-8100 strain as a template, and using primers of SEQ ID NOs: 9 and 10, and primer pairs of SEQ ID NOs: 11 and 12. PCR was performed under the following conditions: denaturation at 94° C. for 5 min, then 30 cycles of 94° C. for 30 sec, 55° C. for 30 sec and 72° C. for 1 min and 30 sec, followed by polymerization at 72° C. for 5 min. The resulting PCR product was cleaved with Smal restriction enzyme to obtain a linear pDZ vector, and the homologous sequence of the terminal 15 base between DNA fragment was fused and cloned using an In-Fusion enzyme, thereby the vector pDZ-leuA (P247C, R558H, G561D) which comprises a leuA mutation encoding a LeuA variant in which arginine, which is the 558th amino acid, is substituted with histidine, and glycine, which is the 561th amino acid, is substituted with aspartic acid in the LeuA amino acid sequence of the wild-type strain, and proline (Pro), the 247th amino acid of LeuA, is substituted with cysteine (Cys), was constructed.

### Example 4-3. Introduction and Evaluation of LeuA Mutant (P247C) in CLJ-8100 Strain

CJL-8100, which is an L-leucine-producing strain, was transformed with the pDZ-leuA (P247C, R558H, G561D) vector produced in Example 4-2, and strains in which the vector was inserted on the chromosome by recombination of homologous sequences were selected in a medium containing 25 mg/L of kanamycin. The selected primary strain was again subjected to a secondary cross-over, and the strain into which the mutation of the target gene was introduced was selected. Whether or not to introduce the leuA gene mutation of the finally transformed strain was confirmed through nucleotide sequence analysis after PCR was performed using the primers of SEQ ID NO: 9 and SEQ ID NO: 15 under the following conditions: 94° C. for 5 min, then 30 cycles of 94° C. 30 sec/55° C. for 30 sec/72° C. for 90 sec, followed by 72° C. for 5 min. As a result of nucleotide sequence analysis, a leuA mutation encoding a LeuA variant (P247C, R558H, G561D) in which arginine, which is the 558th amino acid of the LeuA protein, is substituted with histidine, glycine, which is the 561th amino acid, is substituted with aspartic acid, and proline (Pro), which is the 247th amino acid was substituted with cysteine (Cys), by substituting G, which is the 1673th nucleotide of the leuA gene with A; GC, which is the 1682th and 1683th nucleotides, with AT; and CC, which is the 739th and 740th nucleotides with TG in the strain chromosome, was introduced into the strain. The produced CJL8100_leuA_P247C was named ‘CA13-8105’, and was deposited to the Korean Culture Center of Microorganisms (KCCM), which is an international depositary authority under the Budapest Treaty, on Apr. 29, 2020, under the given deposit number. Amino acid sequence of LeuA variant (P247C, R558H, G561D) comprising the three mutations and the nucleotide sequence of the leuA variant encoding the same are the same as SEQ ID NO: 5 and SEQ ID NO: 6, respectively.

ATCC13032, the produced CJL-8100, and CA13-8105 strains were evaluated for the L-leucine producing ability. Specifically, flask culture was performed in the same manner as in Example 2-2, and after completion of the culture, the L-leucine production amount of the parent strain and the mutant strain was measured by HPLC, and the results are shown in Table 8 below.

TABLE 8

| Strain name | L-leucine (g/L) |
|---|---|
| ATCC13032 | 0.87 |
| ATCC13032_leuA_(R558H, G561D): CJL-8100 | 2.71 |
| CJL8100_leuA_P247C: CA13-8105 | 3.52 |

As shown in Table 8, the L-leucine-producing strain, Corynebacterium glutamicum CJL8100 improved an L-leucine producing ability by about 130% as compared with the parent strain ATCC13032. CA13-8105 strain, which additionally introduced the leuA_P247C mutation in CJL8100 strain, improved L-leucine producing ability by about 150% as compared with the parent strain CJL8100. Through the above results, it can be confirmed that the amino acid at position 247 in the amino acid sequence of the LeuA protein is an important position for L-leucine production activity.

### Example 4-4. Measurement of Isopropylmalate Synthase Activity in the Strain Introduced with LeuA Variant

In order to measure the activity of isopropylmalate synthase in CJL-8100 and CA13-8105, which are L-leucine producing strains produced in Example 4-3, the experiment was performed by the following method.

The strains (CJL-8100, CA13-8105) and wild type ATCC13032 were respectively inoculated into a 250 ml corner-baffled flask containing 25 ml of each seed medium (production medium in Table 2) using a platinum loop, and then was subjected to shaking culture at 30° C. with a shaking rate of 200 rpm for 16 hours. After completion of the culture, the culture solution was centrifuged, the supernatant was discarded, and the pellet was suspended and washed with a lysis buffer solution, and the cells were disrupted with a bead homogenizer. The protein quantification in the lysate is based on Bradford assay method, and a lysate containing 100 μg/ml of protein was used. The activity of the isopropylmalate synthase enzyme was measured by measuring the change in absorption at 412 nm due to thionitrobenzoate (TNB) formed from DTNB (5,5′-dithiobis-(2-nitrobenzoic acid), Ellman's reagent) by reduction using the produced CoA, at this time.

The results of measuring the activity of isopropylmalate synthase in each strain are shown in Table 9 below.

TABLE 9

| | Relative isopropyl- |
|---|---|
| | malatesynthase activity |
| Strain | (%) |
| ATCC13032 | 100 |
| ATCC13032_leuA_(R558H, G561D): CJL-8100 | 113 |
| CJL8100_leuA_P247C: CA13-8105 | 121 |

Next, in order to confirm the degree of release of the feedback inhibition of the above enzyme to leucine, the CoA produced when using a solution containing 100 μg/ml of protein was measured under the condition to which 2 g/l of leucine was added, thereby measuring the activity of isopropylmalate synthase. The results of measuring the activity of isopropylmalate synthase in each strain are shown in Table 10 below.

TABLE 10

| | Leucine 0 g/l | Leucine 2 g/l |
|---|---|---|
| | Relative isopropylmalate | |
| Strain | Synthase activity (%) | |
| ATCC13032 | 100 | 36 |
| ATCC13032_leuA_(R558H, G561D): | 100 | 78 |
| CJL-8100 | | |
| CJL8100_leuA_P247C: CA13-8105 | 100 | 88 |

As shown in Tables 9 and 10, it was confirmed that L-leucine-producing strains CJL-8100 and CA13-8105 transformed with LeuA mutant expression vector increased an isopropylmalate synthase activity by 1.13 times and 1.21 times, respectively, as compared with the control, Corynebacterium glutamicum ATCC 13032. In addition, it was confirmed that the L-leucine-producing strains maintained the isopropylmalate synthase enzyme activity at 78% and 88%, respectively, even under the condition to which 2 g/l of leucine was added, showing that the feedback inhibition by leucine was released.

From the foregoing, a skilled person in the art to which the present invention pertains will be able to understand that the present invention may be embodied in other specific forms without modifying the technical concepts or essential characteristics of the present invention. In this regard, the exemplary embodiments disclosed herein are only for illustrative purposes and should not be construed as limiting the scope of the present invention. On the contrary, the present invention is intended to cover not only the exemplary embodiments but also various alternatives, modifications, equivalents, and other embodiments that may be comprised within the spirit and scope of the present invention as defined by the appended claims.

### [Accession Number]

Name of Depository Authority: Korea Culture Center of Microorganisms
Accession number: KCCM12709P
Date of the deposit: 20200429

1. A polynucleotide that encodes a mutated polypeptide with isopropylmalate synthase activity,
wherein the mutated polypeptide comprises an amino acid sequence at least 90% identical to amino acid residues 36-616 of SEQ ID NO: 1 that is a wild-type sequence,
wherein the mutated polypeptide has an amino acid residue other than proline at a position corresponding to position 247 in the amino acid sequence of SEQ ID NO: 1, and
wherein the amino acid residues 36-616 are set forth in SEQ ID NO: 16.

2. The polynucleotide of claim 1, wherein the position corresponding to position 247 in the amino acid sequence of SEQ ID NO: 1 is substituted with cysteine.

3. The polynucleotide of claim 1, wherein the mutated polypeptide comprises the amino acid sequence of SEQ ID NO: 3.

4. The polynucleotide of claim 1, wherein the mutated polypeptide has an amino acid residue other than arginine at a position corresponding to position 558 in the amino acid sequence of SEQ ID NO: 1.

5. The polynucleotide of claim 4, wherein the mutated polypeptide comprises the amino acid sequence of SEQ ID NO: 5.

6. The polynucleotide of claim 1, wherein the mutated polypeptide has an amino acid residue other than a glycine at a position corresponding to position 561 in the amino acid sequence of SEQ ID NO: 1.

7. The polynucleotide of claim 1, wherein the mutated polypeptide has
an amino acid residue other than an arginine at a position corresponding to position 558 in the amino acid sequence of SEQ ID NO: 1, and
an amino acid residue other than a glycine at a position corresponding to position 561 in the amino acid sequence of SEQ ID NO: 1.

8. The polynucleotide of claim 1, wherein the mutated polypeptide has histidine at a position corresponding to position 558 in the amino acid sequence of SEQ ID NO: 1.

9. The polynucleotide of claim 1, wherein the mutated polypeptide has aspartic acid at a position corresponding to position 561 in the amino acid sequence of SEQ ID NO: 1.

10. The polynucleotide of claim 1, wherein the mutated polypeptide has
histidine at a position corresponding to position 558 in the amino acid sequence of SEQ ID NO: 1; and
aspartic acid at a position corresponding to position 561 in the amino acid sequence of SEQ ID NO: 1.

11. The polynucleotide of claim 1, comprising an amino acid sequence at least 98% identical to the amino acid sequence of SEQ ID NO: 16.

12. The polynucleotide of claim 1, comprising an amino acid sequence at least 95% identical to the amino acid sequence of SEQ ID NO: 1.

13. The polynucleotide of claim 1, comprising an amino acid sequence at least 99% identical to the amino acid sequence of SEQ ID NO: 1.

14. A microorganism comprising at least one selected from the group consisting of the polynucleotide of claim 1 and a polypeptide encoded by the polynucleotide.

15. The microorganism of claim 14, wherein the microorganism has an ability to produce an L-leucine.

16. The microorganism of claim 14, wherein the microorganism is the genus Corynebacterium.

17. The microorganism of claim 16, wherein the microorganism of the genus Corynebacterium is Corynebacterium glutamicum.

18. A method for producing L-leucine comprising a step of culturing the microorganism of claim 14 in a medium

19. The method for producing L-leucine of claim 18, wherein the method further comprises a step of collecting L-leucine from the cultured medium or microorganism.
